(19) United States
(12) Patent Application Publication
(10) Pub. No.: US 2013/0072772 A1
(43) Pub. Date: Mar. 21, 2013

(54) Electrode Catheter Device

(71) Applicants: Stephan Fandrey, Affoltern am Albis (CH); Michael Diebold, Berlin (DE); Sabine Hoffmeister, Hamm (DE); Ingo Weiss, Berlin (DE)

(72) Inventors: Stephan Fandrey, Affoltern am Albis (CH); Michael Diebold, Berlin (DE); Sabine Hoffmeister, Hamm (DE); Ingo Weiss, Berlin (DE)

(73) Assignee: BIOTRONIK SE & CO. KG, Berlin (DE)

(21) Appl. No.: 13/617,681

(22) Filed: Sep. 14, 2012

Related U.S. Application Data
(60) Provisional application No. 61/537,073, filed on Sep. 21, 2011.

(51) Int. Cl. A61B 5/04; A61N 1/05

(52) U.S. Cl. 600/373; 607/116

(57) ABSTRACT

An electrode catheter device, including a conductor structure having at least one electrode, current measuring means for measuring a current induced in the conductor structure by an external electromagnetic field, wherein the current measuring means is designed for the local current measurement, in particular, current intensities and phases in the at least one electrode or a predetermined region of the conductor structure, and is connected to a signal transmission channel that has no interaction with the external electromagnetic field.

### CROSS-REFERENCE TO RELATED APPLICATIONS

This patent application claims the benefit of co-pending U.S. Provisional Patent Application No. 61/537,073, filed on Sep. 21, 2011, the disclosure of which is hereby incorporated by reference in its entirety.

### TECHNICAL FIELD

The present invention relates to an electrode catheter device, which comprises a conductor structure having at least one electrode. It further relates to an electromedical apparatus which comprises such an electrode catheter device.

### BACKGROUND

Such electrode catheter devices are, in particular, stimulation electrode leads (at times also referred to in short as “electrodes”) of cardiac pacemakers or shock electrode leads of implantable defibrillators, but they may also be catheters having an elongated conductive structure.

Medical implants, such as the pacemakers and defibrillators mentioned above, frequently have an electrical connection to the interior of the patient's body. Such a connection is used to measure electrical signals for stimulation, for obliteration, and/or for the defibrillation of body cells. This connection is often configured as an oblong electrode. At present, electrical signals are transmitted between the implant and the electrode contacts (e.g., tip, rings, HV shock coils, sensors, or the like) by way of materials having good electrical conductivity.

When a system comprising an implant and an electrode is exposed to strong interference fields (e.g., EMI, MRI, etc.), undesirable error behavior may occur, especially heating of parts of the system or electrical malfunctions (for example, resets). Heating may result in damage to body tissue or organs when the heated parts have direct contact with the tissue/organs. This is notably the case with the electrode tip.

The cause of the undesirable error behavior is the interaction of the field with the oblong lead structure of the electrode: The electrode acts as an antenna and receives energy from the surrounding fields. At the leads used for treatment, the antenna can give off this energy distally by way of the electrode contacts (e.g., tip, ring, etc.) to the tissue, or proximally to the implant.

The same problems also occur with other oblong conductive structures, the proximal end of which is not necessarily connected to an implant (such as, for example, with catheters, temporary electrodes, and the like).

The most critical instance of interference is the resonance effect, which can be minimized, for example, by shielding or the use of chokes, and prevented by optical or inductive decoupling. The aforementioned use of chokes is described, for example, in “Reduction of Resonant RF Heating in Intravascular Catheters Using Coaxial Chokes” Magn Reson Med. 43(4);615-9, April 2010. The option of optical decoupling is described, for example, in U.S. Pat. No. 6,925,322 or in “Magnetic Resonance Safety Testing of a Newly-Developed Fiber-Optics Cardiac Pacing Lead”, J Magn Reson Imaging. 16(1); 97-103 July 2002. The option of inductive decoupling is described, for example, in “Multifunctional Interventional Devices for MRI: A Combined Electrophysiology/MRI Catheter” Magn Reson Med. 47(3);594-600, March 2002, or “Feasibility of Real-Time Magnetic Resonance Imaging for Catheter Guidance in Electrophysiology Studies” Circ 2008:118-223-227.

Shielding or the use of sheath current chokes can achieve an effective reduction in the RF-induced effects. However, for this purpose every possible position and posture in the MRI and in the patient must be taken into consideration. Accidental RF coupling can thus never be excluded.

The Applicant develops catheter prototypes in which a metallic conductor structure is replaced with carbon fibers, or in which non-magnetic materials are employed; with respect to these options, refer also to “Feasibility of Real-Time MRI With a Novel Carbon Catheter for Interventional Electrophysiology”, 2009/3 Circ 2009:2:258-267 or “Interactive Real-Time Mapping and Catheter Ablation of the Cavotricuspid Isthmus Guided by Magnetic Resonance Imaging in a Porcine Model”, 2009/11 Eur. Heart Journal.

Moreover, reference is made to publications that relate to an MR probe integrated in a catheter tip and, more specifically, to S. Fandrey, S. Weiss, J. Muller “Development of an Active Intravascular MR-Device with an Optical Transmission System”, IEEE Transactions on Medical Imaging 2008; 27; 1723-1727 and “Aktive Magnetresonanz-Sonde in Mikrosystemtechnik auf Basis einer optischen Signalübertragung für die minimal-invasive Chirurgie [Active magnetic resonance probe in microsystem technology based on optical signal transmission for minimally invasive surgery]”, Verlag Dr. Hut publisher, Munich 2010. Therein, the integration of an electro-optical transducer, including an optical power supply in a 6F catheter, is described. This probe allows for general MR signal transmission for localization and imaging purposes in a vascular system.

The possible interferences between the MR device and critically long conductor structures (such as EP catheters, for example) and known approaches that relate to safety have already been scientifically discussed. In addition, the publication by M. G. Zanchi, R. Venook, J. Pauly, G Scott “An Optically Coupled System for Quantitative Monitoring of MRI-Induced RF Currents Into Long Conductors”, IEEE Transaction On Medical Imaging, Vol. 29, No. 1, January 2010, scientifically discusses a solution for measuring MRI-induced RF currents in long conductors by means of a measuring system using optical coupling.

The present invention is directed toward overcoming one or more of the above-identified problems.

It is the object of the present invention to create an improved electrode catheter device which is equipped so that it can more precisely detect interferences that may develop with the use in strong external electromagnetic fields, so as to be able to take exactly defined counter-measures.

### SUMMARY

In order to use the proposed electrode catheter device or electromedical apparatus, a one-time calibration measurement for a specific catheter or electrode system is required. During this calibration measurement, the correlation between the RF current and the tissue heating is recorded by means of the current sensor and a temperature sensor. In this way, an estimation can be made later, during the real operation, as to which currents are still within the permissible range and which currents cause damaging RF heating.

RF-induced currents caused by MRI can lead to tissue heating that is dangerous for the patient. The current sensor detects currents that exit the tip electrode and/or the ring electrodes. Tissue heating can be counter-acted with the detected current. This can be done by either deactivating or adapting the RF output of the MRI or by additional cooling of the catheter tip.

This object is achieved by an electrode catheter device having the characteristics of the independent claim(s). Advantageous refinements of the present invention are the subject matter of the dependent claims including, without limitation, an electromedical apparatus.

The solution according to the present invention improves the safety and/or enables the use of a catheter with electrodes or cardiac pacemaker/ICD electrodes in the MRI. The sensors allow a dangerous situation to be detected quickly and make it possible to take direct counter-measures as they relate to the RF-induced tissue heating.

Generally speaking, MRI as an imaging method has several advantages over X-rays, in particular with minimally invasive surgeries or during the imaging of organs and soft tissue. An MR-safe ablation catheter, for example, could be used for the real-time imaging of lesions shortly after or during ablation. Older lesions can also be represented with the MR imaging method. In addition, the MR imaging method simplifies navigation. On an overall basis, a higher success rate during ablation procedures is thus to be expected. Moreover, no ionizing radiation exists in MR imaging methods, which can be considered an added advantage over X-rays.

The present invention proposes the use of current measuring means in the electrode catheter device which are designed for local current measurement of, in particular, the current intensity and phase in the electrode(s) or in another predetermined region of the conductor structure. It further includes the idea that these current measuring means are connected to a signal transmission channel which does not interact with the external field.

In particular, the safe operation of the catheter can thus be assured and dangerous situations can be detected. With the integration in a cardiac pacemaker/ICD electrode, the current sensor can detect the RF-induced currents and send a warning through the implant via a communication channel. In addition to the aforementioned cardiac pacemaker and ICD electrode lines, the present invention can also advantageously be used in catheter assemblies for kidney or tumor ablation, and in assemblies for neurostimulation. Even electrode lines that are used solely as sensors, or lines that are utilized for transmitting sensor signals and for transmitting therapy currents, are a useful field of application of the invention (beyond the widely common use therefore in cardiac pacemaker assemblies).

In one embodiment of the present invention, the current measuring means are associated with an electric/mechanical transducer for converting the signal into a mechanical signal, and the signal transmission channel is designed for mechanical signal transmission. In an alternative embodiment, the current measuring means are associated with an electric/optical transducer for converting the signal into an optical signal, and the signal transmission channel is designed for optical signal transmission. According to another embodiment, the current measuring means are associated with an electric/thermal transducer for converting the signal into a thermal signal, and the signal transmission channel is designed for thermal signal transmission.

According to a further concept for substantially suppressing interferences in the transmission channel, an electric/electric transducer or modulator is associated with the current measuring means for converting the output signal of the current measuring means into, or for modulation onto, electromagnetic waves in a frequency range which does not significantly interfere with a frequency spectrum of the external field, and the signal transmission channel is designed for electromagnetic signal transmission. To this end, in particular, the signal transmission channel can comprise a wireless transmission section. This reliably prevents inductive coupling of interferences, but must be accompanied by measures to prevent interference of the signal transmission waves with interfering waves from the external field.

This is possible particularly efficiently if the electric/electric transducer or modulator is associated with coding means for coding the output signal of the current measuring means so as to reduce interfering influences from the external electromagnetic field. The coding of data signals has proven, not only in mobile radio technology, as an extremely effective measure for safeguarding a substantially interference-free and reliable signal transmission in environments subject to high levels of interference, and a person skilled in the art is familiar with suitable coding methods. Thus, a detailed description is not necessary.

Independently of the aforementioned measures, but also in combination therewith, the signal transmission channel can comprise an electric conductor which, in coordination with the geometric design of the conductor structure, is produced from such a material and/or shaped such and/or wired such that only low coupling with the external electromagnetic field and the conductor structure takes place.

According to a further embodiment of the present invention, the current measuring means are directly associated with an electric conductor as the electric signal transmission channel, or as part of the same, wherein the current measuring means and the associated conductor are in particular, integrally designed.

In a further embodiment of the present invention, the current measuring means are designed as a passive or energy self-sufficiently operating measuring means. They can be operated, for example, utilizing the body heat of a living being where the electrode catheter device is introduced. In addition, movements of the living being can be utilized to supply the current measuring means with power. The utilization of energy from an external interference field for operating the current measuring device is also possible.

In an alternative embodiment, the current measuring means are designed as active measuring means and connected to an energy transmission channel. Analogously with the signal transmission channel, the integration of a wireless transmission section may be provided for. In addition, the energy transmission channel can, of course, comprise an electric conductor, in particular, an electrode conductor or ion conductor. On the other hand, the energy transmission channel can comprise means for the mechanical, in particular, hydraulic or pneumatic, energy transmission, or means for the optical energy transmission, in particular, an optical fiber.

With respect to the aforementioned active design of the current measuring means, the measurement or therapy device of the proposed electromedical apparatus can comprise an energy source that can be connected to the energy transmission channel of the electrode catheter device for supplying the current measuring means with energy.

In a preferred embodiment of the inventive apparatus, the measurement or therapy device comprises an operating control device that is connected on the input side to the evaluation means for controlling a measurement and/or therapy process as a function of the evaluation result of the output signal of the current measuring means. In principle, the object pursued by the present invention can also be achieved by influencing the measurement or therapy process, or the parameters thereof, externally in relation to the actual measurement or therapy device, for example, by suitably designing a central, subsequent evaluation or by influencing the patient outside of the actual measurement or therapy channel.

Aspects of embodiments of the present invention will additionally become apparent from the following list:
The current sensor is a transducer which forwards a variable that is proportional to the current (or that, in general, represents a function thereof) to an evaluation unit.
The current sensor is connected in series to the functional line and directly detects the current; it is designed, for example, as a resistor or, in general, as an impedance (which is to say also as a capacitor or coil or transmitter) and at the ends thereof generates a voltage drop that is proportional to the current. The transmitted measurement signal is thus a voltage. It is also possible to use a non-linear resistor, which codes the signal amplitude, for example, logarithmically.
The current sensor converts the current flowing through it into an acoustic/mechanical (piezo/quart), optical (LED), pneumatic/hydraulic or temperature signal.
The current sensor converts the current flowing through it into a wave that can be transmitted wirelessly (in particular, also via the body), for example, an electromagnetic wave (in a frequency range that does not interfere with the interference fields), or acoustic wave (in a frequency range that does not interference with the gradients).
The current sensor indirectly converts the current by detecting the magnetic field, or the magnetic flow, generated by the current. The advantage: The function conductor does not have to be separated, and reliability problems related to the contacting technology are prevented.
The current sensor comprises a primary transducer (for example, current into voltage) and a secondary transducer, which converts, for example, this voltage in a different variable that is better suited for the transmission to the evaluation unit, for example, an acoustic/mechanical (piezo/quart), optical (LED), pneumatic/hydraulic or temperature signal.
The energy is supplied electrically (electron conductor, ion conductor, electrolyte tube), mechanically/acoustically (piezo), optically (laser/light), or hydraulically/pneumatically.
The magnetic field sensor is a coil (in general, a second conductor that couples inductively to the function conductor).
The magnetic field sensor is a Hall sensor, or GMR sensor.
For improved coupling of the sensor to the function conductors, these conductors are, or at least one of them is, suitably designed at the location of the current measurement; for example, even if the function conductor has a linear progression otherwise, it is coiled here, and the sensor coil forms the secondary winding of this transmitter. In contrast, if the function conductor has a linear progression, the sensor can be a toroidal coil (e.g., Rogowski coil), for example.
For improved coupling of the sensor to the function conductor or conductors, the conductor or at least one of them is provided with suitable material properties at the location of the current measurement; for example, the sensor conductors and/or function conductors are provided with a highly conductive coating, so that the resulting transmitter has lower losses.
So as to reduce the coupling to the function conductor or external interference, the feed line from the sensor to the evaluation unit is designed as an optical conductor, acoustic conductor, or hydraulic/pneumatic conductor.
So as to reduce the coupling to the function conductor or external interference, the feed line from the sensor to the evaluation unit is designed as an electric conductor, wherein the coupling is reduced by suitably shaping and/or guiding the conductors (for example, such that they inductively couple to a minimal extent, and preferably linearly extend to the greatest extent possible, for example, extending linearly in an otherwise coiled structure of the function conductors).
So as to reduce the coupling to the function conductor or external interference, the feed line from the sensor to the evaluation unit is designed as an electric conductor, wherein the coupling is reduced by suitable materials, for example, high-resistance wires, ion conductors having high resistance (accordingly low-conductive electrolyte), or the like.
In a preferred embodiment, the sensor and the feed line to the evaluation unit comprise the same material and have no connecting technology. (Implementation example: The sensor coil is wound of one and the same piece of resistance wire of which the feed line is made.)
The coding can be implemented, for example, as FM PWM, phase or polarization coding.

Further features, aspects, objects, advantages, and possible applications of the present invention will become apparent from a study of the exemplary embodiments and examples described below, in combination with the figures, and the appended claims.

### DESCRIPTION OF THE DRAWINGS

Advantages and functional characteristics of the invention will additionally become apparent hereafter from the sketch-like description of exemplary embodiments based on the figures, wherein it is shown in:

FIGS. 1A-1B are two variants of an embodiment of the present invention as schematic cross-sectional views;

FIG. 1C is a schematic diagram of an evaluation unit associated with the electrode catheter devices according to FIG. 1A or 1B;

FIG. 1D is a detailed view of a current sensor used in the electrode catheter device according to FIG. 1A or 1B;

FIG. 1E is a schematic diagram of an embodiment of the measuring element used in the current sensor;

FIG. 1F is a schematic diagram of a measuring element that can be used as an alternative embodiment;

FIG. 2 is a schematic diagram of an electric-optical modulator that can be used in an electrode catheter device according to the present invention; and

FIG. 3 is a functional block diagram of an electromedical assembly according to the present invention.

### DETAILED DESCRIPTION

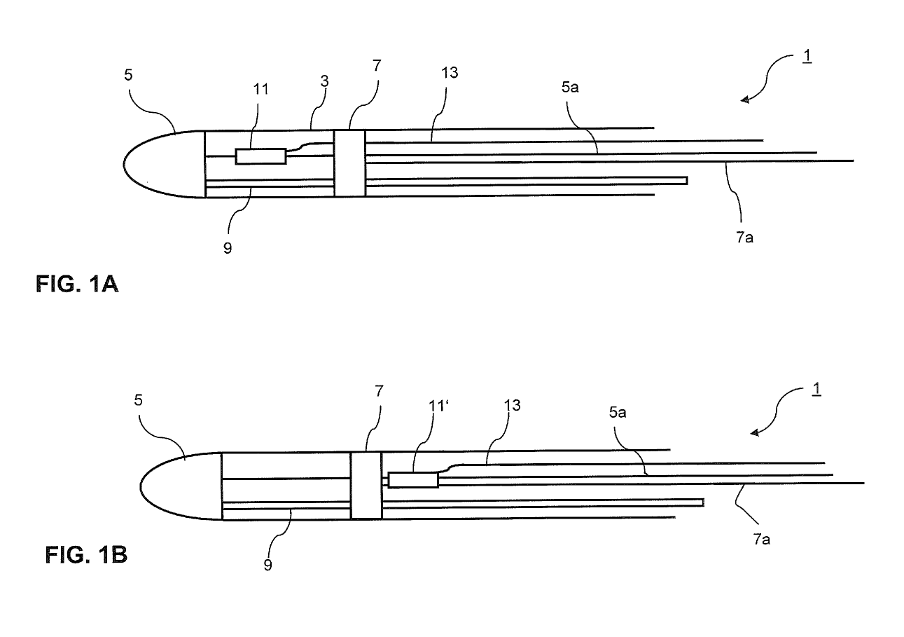

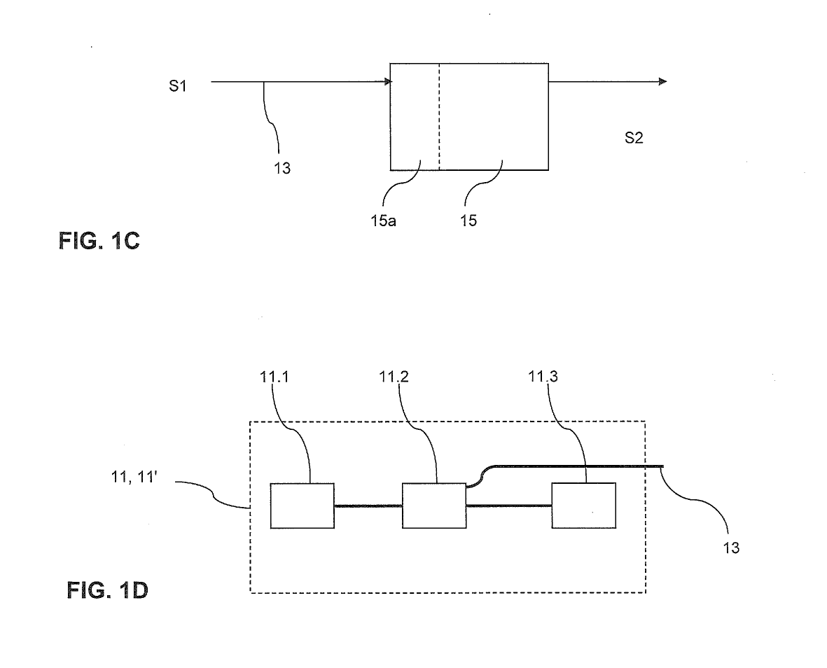

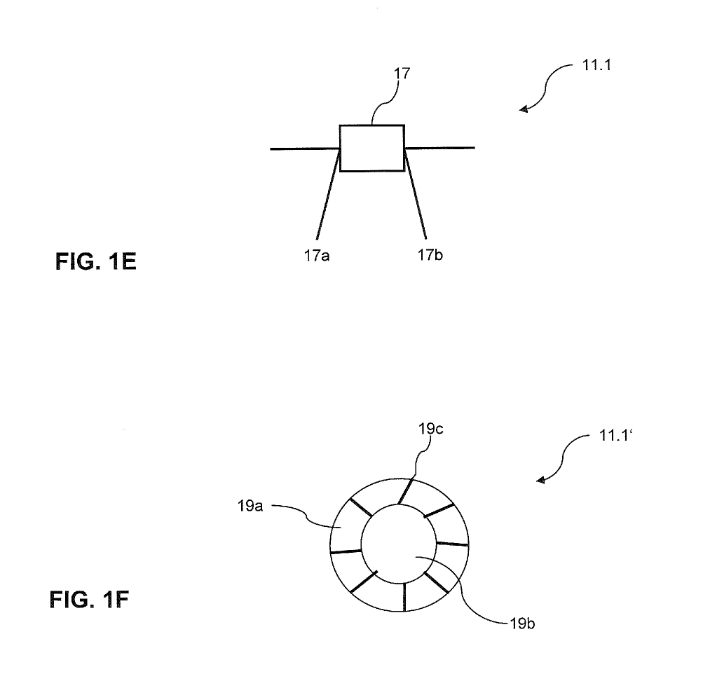

FIG. 1A is a schematic illustration of the distal end of a pacemaker electrode line 1 which, in a line body 3, comprises a tip electrode 5 and a ring electrode 7 that is disposed at a distance therefrom, along with related feed lines 5a and 7a and a flushing channel 9. A current sensor 11, to which an optical waveguide 13 is connected as the transmission channel, is provided adjacent to the tip electrode 5 for measuring the current in the feed line 5a to the tip electrode 5. A modified electrode line 1′ according to FIG. 1B basically has the same design; however; it contains a current sensor 11′ that is designed for the current measurement in the two feed lines 5a, 7a and is accordingly positioned in a different location.

FIG. 1C basically shows the end of the signal transmission channel which, is to say an evaluation unit 15 that is connected to the optical waveguide 13, an optical/electric transducer 15a for reconverting the measurement signal that was transmitted optically being provided at the input of the evaluation unit 15, and the unit 15 generating from a measurement signal S1 an evaluation result signal S2 that can be utilized for controlling a measurement or therapy process.

The evaluation unit 15 converts the optical signal back into an electric signal and filters the signal according to frequencies. For example, a current in the kHz range, which is of therapeutic benefit (for example, during ablation), can be easily differentiated from an RF current in the MHz range that is induced by the MRI. When an RF-induced current in the MHz range is detected, the evaluation unit 15 can emit appropriate warning signals and thus prevent undesirable tissue heating. These signals can be used directly for counter-measures (e.g., closed loop). For example, the RF output can be deactivated or reduced for the imaging process. As an alternative, the resulting heating can be cooled, having knowledge of the current, by way of the rinsing of the catheter. When the relationship between the exiting RF current and the tissue heating is known, the quantity and flow of the rinsing can be automatically controlled for cooling depending on the situation. In addition, the evaluation unit 15 will directly detect the failure of the optical current sensor, whereby reliable observation of the induced RF current is ensured. This is possible because the optical modulator operates the laser power in the CW mode. Failure of the laser diode can thus be detected directly by the receiver on the evaluation unit 15 based on the failure of the optical power in the optical waveguide.

A possible design of the current sensor 11 or 11′ of FIG. 1A or 1B, respectively, is shown schematically in FIG. 1D. The current sensor according to this diagram comprises three functional blocks, which is to say a measuring element 11.1 (in the narrower sense), an electric/optical modulator 11.2, and a power supply block 11.3 for the modulator 11.2.

FIG. 1E is a schematic illustration of an embodiment of the measuring element 11.1 as a resistor 17 that is introduced in the feed line 5a according to FIG. 1A and comprises related lines 17a, 17b for capturing a voltage which drops over the resistor 17 and can be used as a measurement signal. As an alternative embodiment of the current sensor 11.1′, FIG. 1F shows a toroidal coil comprising a coil body 19a, which has a line feed through 19b for the line on which the current flow is to be measured to pass through, and a coil winding 19c. For practical use, the coil should be shielded from B1 fields of the MRI. The shield should be slotted along the inner ring, whereby a Rogowski coil is obtained, for example. The coil is thus sensitive to current-carrying conductors that pass through and provides an effective shield to fields acting from the outside.

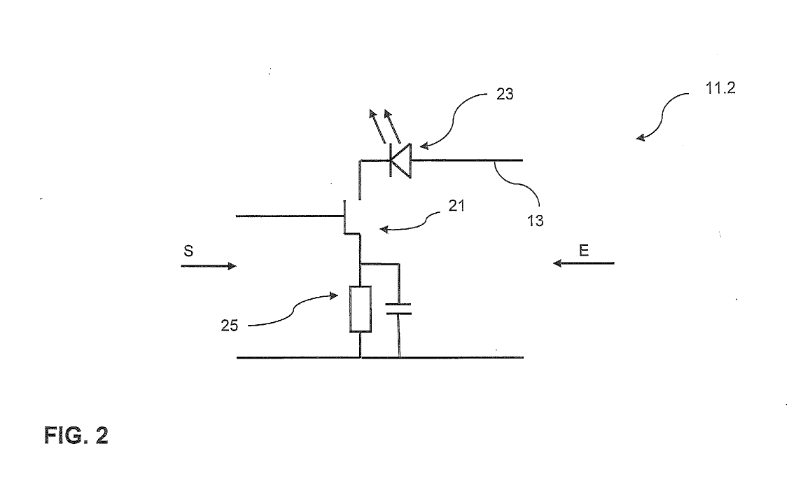

FIG. 2 shows—again only schematically—an implementation of the electric/optical modulator 11.2 from FIG. 1D comprising a transistor 21, a laser diode 23 and RC member 25. The arrow S denotes the signal input, and the arrow E denotes the power supply side of the modulator assembly. The power supply can be implemented, for example, as an optical power supply via a photovoltaic element, or as an electric power supply via a high-resistance wire, or also in another manner. According to initial findings of the Applicant, high-resistance wires also enable a low-interference power supply and, according to the present state of knowledge, the effort for the integration in the electrode catheter device is lower than with an optical power supply.

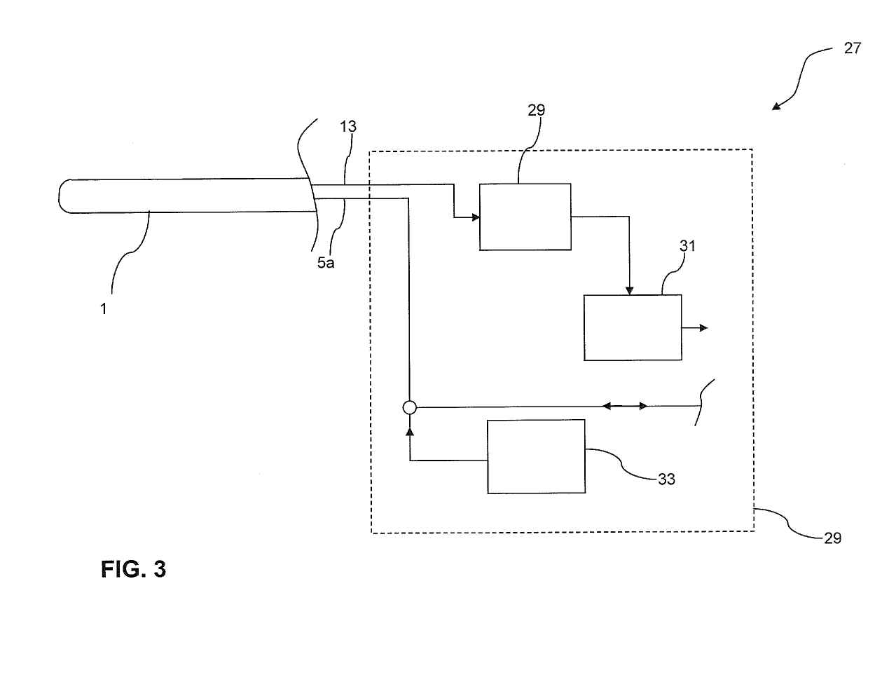

FIG. 3 shows—again schematically in the manner of a function block diagram—the basic design of an electromedical apparatus 27 according to the present invention, here specifically a pacemaker assembly, which comprises a pacemaker line 1 (see FIG. 1A) and a cardiac pacemaker 29 that is adapted according to the present invention. In addition to the standard components of a cardiac pacemaker (not shown here), also integrated is a control block 31 that is connected on the output side to the evaluation block 15 (see FIG. 1C) for influencing the pacemaker therapy as a function of the signals of the current sensor integrated in the pacemaker line, and finally a sensor power supply block 33 which, in this example, is connected on the output side to the electrode feed line 5a. This line at the same time constitutes the power transmission channel of the assembly, which requires suitable decoupling (which can be solved within the scope of the knowledge of a person skilled in the art) with respect to the treatment signals transmitted on the same line.

The implementation of the present invention is not limited to the examples described above and concepts emphasized, but is likewise possible in a plurality of modifications, which are within the scope of standard practice in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. An electrode catheter device, comprising:
a conductor structure having at least one electrode; and
current measuring means for measuring a current induced in the conductor structure by an external electromagnetic field, wherein the current measuring means is designed for the local current measurement, in particular current intensities and phases, in the at least one electrode or a predetermined region of the conductor structure, and wherein the current measuring means is connected to a signal transmission channel that has no interaction with the external electromagnetic field.

2. The electrode catheter device according to claim 1, wherein the current measuring means is associated with an electric/mechanical transducer for converting the electrical signal into a mechanical signal, and wherein the signal transmission channel is designed for mechanical signal transmission.

3. The electrode catheter device according to claim 1, wherein the current measuring means is associated with an electric/optical transducer for converting the electrical signal into an optical signal, and wherein the signal transmission channel is designed for optical signal transmission.

4. The electrode catheter device according to claim 1, wherein the current measuring means is associated with an electric/thermal transducer for converting the electrical signal into a thermal signal, and wherein the signal transmission channel is designed for thermal signal transmission.

5. The electrode catheter device according to claim 1, wherein an electric/electric transducer or modulator is associated with the current measuring means for converting the output signal of the current measuring means into, or for modulation onto, electromagnetic waves in a frequency range which does not significantly interfere with a frequency spectrum of the external electromagnetic field, and wherein the signal transmission channel is designed for electromagnetic signal transmission.

6. The electrode catheter device according to claim 5, wherein the signal transmission channel comprises a wireless transmission section.

7. The electrode catheter device according to claim 5, wherein the electric/electric transducer or modulator is associated with coding means for coding the output signal of the current measuring means so as to reduce interfering influences from the external electromagnetic field.

8. The electrode catheter device according to claim 5, wherein the signal transmission channel comprises an electric conductor, which in coordination with a geometric design of the conductor structure, is produced from such a material and/or shaped such and/or wired such that only low coupling with the external electromagnetic field and the conductor structure takes place.

9. The electrode catheter device according to claim 1, wherein the current measuring means is directly associated with an electric conductor as the electric signal transmission channel, or as part of the same, and wherein the current measuring means and the associated conductor are integrally designed.

10. The electrode catheter device according to claim 1, wherein the current measuring means is designed as a passive or energy self-sufficiently operating measuring means.

11. The electrode catheter device according to claim 1, wherein the current measuring means is designed as an active measuring means and is connected to a power transmission channel.

12. The electrode catheter device according to claim 11, wherein the power transmission channel comprises a wireless transmission section.

13. The electrode catheter device according to claim 11, wherein the power transmission channel comprises an electric conductor, such as an electrode conductor or ion conductor.

14. The electrode catheter device according to claim 11, wherein the power transmission channel comprises means for mechanical, such as hydraulic or pneumatic, power transmission.

15. The electrode catheter device according to claim 11, wherein the power transmission channel comprises means for optical power transmission, such as an optical fiber.

16. An electromedical apparatus comprising an electrode catheter device according to claim 1, and a measuring device and/or therapy device connected thereto and which comprises evaluation means that can be connected to the signal transmission channel of the electrode catheter device for evaluating an output signal of the current measuring means.

17. The apparatus according to claim 16, wherein the measuring device and/or therapy device comprises a power source that can be connected to the power transmission channel of the electrode catheter device for supplying the current measuring means with power.

18. The apparatus according to claim 16, wherein the measuring device and/or therapy device comprises an operating control device that is connected to the evaluation means for controlling a measurement and/or therapy process as a function of the evaluation result of the output signal of the current measuring means.
